(19) United States
(12) Patent Application Publication
(10) Pub. No.: US 2009/0298786 A1
(43) Pub. Date: Dec. 3, 2009

(54) METHODS OF TREATING VIRAL INFECTIONS WITH ANTHRACYCLINE ANTIBIOTICS

(75) Inventors: Irena OSZCZAPOWICZ, Warszawa (PL); Anna BOGUSZEWSKA-CHACHULSKA, Warszawa (PL); Mariusz KRAWCZYK, Raszyn (PL); Malgorzata LUKAWSKA, Warszawa (PL)

Correspondence Address: MATTHIAS SCHOLL, 14781 MEMORIAL DRIVE, SUITE 1319, HOUSTON, TX, 77079, US

(73) Assignees: INSTYTUT BIOTECHNOLOGII I ANTYBIOTYKOW, Warszawa (PL); INSTYTUT BIOCHEMII I BIOFIZYKI POLSKIEJ AKADEMII NAUK, Warszawa (PL)

(21) Appl. No.: 12/537,270

(22) Filed: Aug. 7, 2009

Related U.S. Application Data
(63) Continuation of application No. PCT/PL2008/000010, filed on Feb. 7, 2008.

(30) Foreign Application Priority Data
Feb. 7, 2007 (PL) P.381714

(51) Int. Cl. A61K 31/704; A61P 31/12

(52) U.S. Cl. 514/34

(57) ABSTRACT

A method of treating hepatitis C viral infection or viremia in a patient by administering to the patient an anti-hepatitis C virally effective amount of a compound of Formula 1 or Formula 2. A method for inhibiting the replication of hepatitis C virus by exposing the virus to a hepatitis C viral NS3 protease inhibiting amount of: a compound of Formula 1 or Formula 2. A pharmaceutical composition exhibiting an antihelicase activity containing at least a pharmaceutically-acceptable carrier, diluent or excipient, and a compound of Formula 1 or Formula 2. The pharmaceutical composition may additionally contain a second compound of Formula 1 or Formula 2; epidoxorubicin, doxorubicin, epidaunorubicin, or daunorubicin; and/or another antiviral agent.

### CROSS-REFERENCE TO RELATED APPLICATIONS

This application is a continuation of International Patent Application No. PCT/PL2008/000010 with an international filing date of Feb. 7, 2008, designating the Unites States, now pending, and also claims foreign priority benefits to Polish Patent Application No. P.381714, filed Feb. 7, 2007. The contents of the above-mentioned applications are incorporated herein by reference in their entirety.

### BACKGROUND OF THE INVENTION

1. Field of the Invention

This invention relates to methods of treating viral infections with anthracycline antibiotics.

2. Description of the Related Art

Hepatitis C virus (HCV) infection is one of the most frequent causes of acute or chronic hepatitis. WHO estimates that about 170 million people, 3% of the world's population, are infected with HCV and are at risk of developing liver cirrhosis and/or liver cancer. In the United States, that number is about 4.1 million, or about 1.6% of the U.S. population. At present, the number of deaths per year caused by the HCV virus amounts to between 8,000 and 12,000 worldwide, and in the next few years it will likely double.

HCV infection results in chronic hepatitis in about 80% of cases, and in 4 to 20% of cases ends in cirrhosis within 5 to 20 years of infection. In 1-5% of those chronically infected, primary liver cancer develops (Brass V. et al., International J. Medicinal Sciences 3, 29-34, 2006). Because of HCV interactions with immunological system during chronic infections other diseases may occur, this being an extrahepatic HCV manifestation. There is no vaccine against HCV due to high variability of viral RNA genome.

Methods of treatment of chronic hepatitis C evolve very fast. Conventionally, monotherapy with interferon alpha (IFN) was used, whereas at the end of the 1990's, a more effective combined therapy with IFN and ribavirin was introduced, and further modification of therapy consisted in using pegylated interferon. At present, a valid treatment scheme is a combined therapy with pegylated interferon alpha-2a or alpha-2b and ribavirin, the dosage being dependent on the HCV genotype. In the case of occurrence of distinct adverse reactions, ineffective treatment, or recurrence of virus replication as well as when treating patients under 18 years old most often a monotherapy is used with other interferons, such as natural interferon alpha-n3 or other preparations, e.g. comprising interferon alpha-2a in a Formulation providing prolonged action, optionally combined with ribavirin.

Prior to the treatment, the HCV genotype in a patient should be determined, as its type defines a relevant treatment method, and in cases of infections caused by genotypes 1, 4, 5 or 6 also the concentration of HCV RNA (a degree of viremia) has to determined. The treatment is considered to be effective if after 24 weeks upon the end of therapy, no HCV RNA is detected in blood.

If the pharmacological treatment fails, the only possible therapy method is liver transplantation, considered as one of the most difficult surgical procedures because of the liver position and its extensive blood supply.

As no fully effective and economic drug against various genotypes has heretofore been developed, there is a need for search of new, more effective and economic therapies against HCV. The search is directed to improve the present treatment standard by employing new interferons (interferon beta-1c) or more suitable forms of that drug, such as prolonged-action interferons or new derivatives of ribavirin exhibiting weaker adverse effects.

Another direction of research pertains to a possibility of interference into the HCV replication cycle. Important elements of such action can be different viral proteins—the NS3 protease/RNA helicase/NTP-ase or the RNA-dependent RNA polymerase (Huang M. and Deshpande M., Expert Rev. Antiinfect. Ther. 2, 375-388, 2004).

The NS3 helicase of HCV seems to be one of the best targets of activity of low-molecular inhibitors as an enzyme indispensable for viral multiplication (Lam A. M. and Frick D. N., J. Virology 80, 404-411, 2006). The helicase activity is necessary at the most important stages of viral life cycle, such as replication and translation. Inhibition of helicase activity and increase of double-stranded RNA level will result in stimulation of antiviral cell response (Gordon C. P. and Keller P. A., J. Med. Chem. 48, 1-20, 2005).

Among many examined compound types, for which antiviral activity was observed, there were also certain members of anthracycline antibiotics. It has been found, for example, that these members inhibit activity of enzymes and/or replication of Human immunodeficiency virus (HIV) of (+)RNA genome. From U.S. Pat. No. 5,003,055, a group of daunorubicin derivatives, inhibiting activity of HIV reverse transcriptase, is known. It has been also shown that doxorubicin selectively inhibits replication of HIV in macrophages, but not in lymphocytes (Bergamini A. et al., AIDS Res. Hum. Retroviruses, 8, 1239-1247, 1992), probably acting by blocking reverse transcriptase reaction. In a similar manner, daunorubicin much more effectively inhibits replication of the virus in monocytes than in lymphocytes T (Filion A. et al., Clin. Invest. Med. 16, 339-347, 1993). Recent studies have shown that a bis-anthracycline derivative denoted as WP631, including molecules built from two daunorubicin moieties connected by a p-xylene group, inhibits HIV replication in lymphocytes (PBMC cells) through inhibition of transactivation of viral protein Tat. Because of low therapeutic index, the compound can serve only as a substrate for synthesis of new derivatives (Kutsch W. et al., Antimicrobial Agents and Chemotherapy 48, 1652-1663, 2004).

Moreover, for certain anthracycline antibiotics, such as daunorubicin, doxorubicin, epirubicin, mitoxantrone and nogalamycin, Borowski et al. have shown a significant antihelicase activity (IC₅₀ values are 57.0, 5.0, 0.75, 6.7 and 0.1 μM, respectively) but these compounds also exhibit high toxicity (Borowski P. et al., Antiviral Res. 55, 397-412, 2002). From EP Patent No. 1721614 known is antiviral activity of epirubicin hydrochloride but this antibiotic exhibits similarly high toxicity.

Certain amidinoanthracycline antibiotics are known from Polish Patent No. 186762 and Polish Patent Application Nos. P.372208 and P.378888. These derivatives, prepared by transformation of the amino group at 3′ position of anthracycline antibiotic to an amidino group, exhibit very advantageous biological properties, such as high cytotoxic activity, considerable, up to 60-fold decrease of toxicity as compared to the parent anthracycline antibiotics, including cardiotoxicity, and moreover, contrary to the parent antibiotics, an ability to overcome a barrier of resistance of tumor cells (Wasowska M. et al., Anticancer Res., 26, 2009-2012, 2005 and Wasowska M. et al, Anticancer Res. 25, 2043-2048, 2006).

### BRIEF SUMMARY OF THE INVENTION

In one embodiment of the invention, provided is the unexpected finding that compounds of Formula 1 or Formula 2

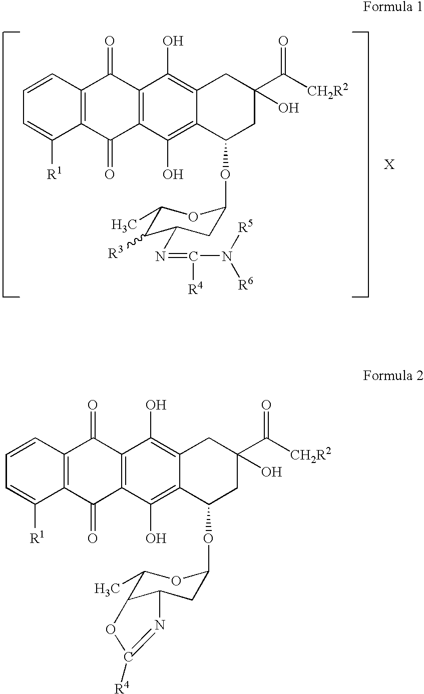

wherein
R¹ is hydrogen or methoxy;
R² is hydrogen or hydroxy;
R³ is hydroxy;
R⁴ and R⁵ are independently hydrogen or methyl, and R⁶ is 1-phenylethyl, or R⁵ and R⁶ taken together with the nitrogen atom to which they are connected are N,N-diethyl, N,N-dipropyl or N,N-dibutyl group, or R⁵ and R⁶ taken together with the nitrogen atom to which they are connected form N,N-1″,4″-tetramethylene, N,N-3″-oxa-1″,5″-pentamethylene, N,N-1″,5″-pentamethylene, N,N-1″,6″-hexamethylene, N,N-1″,7″-heptamethylene, N,N-3″-methylaza-1″,5″-pentamethylene, or 1-indolinyl; and
X is HCl, or is absent, wherein when X is HCl the compound is a hydrochloride salt and when X is absent the compound is a free base;

are strong inhibitors of helicase of hepatitis C virus (HCV), and considerably stronger than daunorubicin, doxorubicin, epidaunorubicin, or epidoxorubicin.

In another embodiment of the invention, provided is a method of treating hepatitis C viral infection in a patient comprising administering to the patient an anti-hepatitis C virally effective amount of: (a) a compound of Formula 1 or Formula 2, or (b) a pharmaceutical composition comprising a pharmaceutically-acceptable carrier, diluent or excipient and a compound of Formula 1 or Formula 2.

In another embodiment of the invention, provided is a method of treating viremia in a patient comprising administering to the patient an anti-virally effective amount of: (a) a compound of Formula 1 or Formula 2, or (b) a pharmaceutical composition comprising a pharmaceutically-acceptable carrier, diluent or excipient and a compound of Formula 1 or Formula 2.

In another embodiment of the invention, provided is a method for inhibiting the replication of hepatitis C virus comprising exposing the virus to a hepatitis C viral NS3 protease inhibiting amount of: (a) a compound of Formula 1 or Formula 2, or (b) a pharmaceutical composition comprising a pharmaceutically-acceptable carrier, diluent or excipient and a compound of Formula 1 or Formula 2.

A method of treating a viral infection in general in a patient comprising administering to the patient an anti-hepatitis C virally effective amount of: (a) a compound of Formula 1 or Formula 2, or (b) a pharmaceutical composition comprising a pharmaceutically-acceptable carrier, diluent or excipient and a compound of Formula 1 or Formula 2.

In another embodiment of the invention, provided is a method for inhibiting helicase activity comprising: exposing the virus to a hepatitis C viral NS3 protease inhibiting amount of: (a) a compound of Formula 1 or Formula 2, or (b) a pharmaceutical composition comprising a pharmaceutically-acceptable carrier, diluent or excipient and a compound of Formula 1 or Formula 2.

In another embodiment of the invention, provided is a method for inhibiting hepatitis C virus in vitro comprising contacting a sample in need of such treatment with (a) a compound of Formula 1 or Formula 2, or (b) a pharmaceutical composition comprising a pharmaceutically-acceptable carrier, diluent or excipient and a compound of Formula 1 or Formula 2.

In another embodiment of the invention, provided is a method of inhibiting hepatitis C virus in a patient, comprising administering an effective amount of (a) a compound of Formula 1 or Formula 2, or (b) a pharmaceutical composition comprising a pharmaceutically-acceptable carrier, diluent or excipient and a compound of Formula 1 or Formula 2.

In another embodiment of the invention, provided is a method of inhibiting hepatitis C virus (HCV) replication in cells infected with HCV comprising: contacting the cells with an effective amount of (a) a compound of Formula 1 or Formula 2, or (b) a pharmaceutical composition comprising a pharmaceutically-acceptable carrier, diluent or excipient and a compound of Formula 1 or Formula 2.

In a class of any of these embodiments, the patient is an animal, particularly a mammal, and more particularly a human.

In a class of any of these embodiments, R³ is axial or equatorial.

In a class of any of these embodiments, the dose of a compound of Formula 1 or Formula 2, or a corresponding dose of a pharmaceutical composition comprising a compound of Formula 1 or Formula 2 is between about 0.005 and about 1.5 mg/kg, corresponding to between about 0.25 and about 75 mg/m². This dose causes an effective inhibition of helicase of hepatitis C virus (HCV), hepatitis C viral infection, hepatitis C virus (HCV) replication, or viremia.

In a class of any of these embodiments, the compound of Formula 1 or Formula 2 is:
3′-deamino-3′-(N,N-1″,4″-tetramethyleneformamidino)-daunorubicin hydrochloride;
3′-deamino-3′-(N,N-1″,4″-tetramethyleneformamidino)-daunorubicin;
3′-deamino-3′-(N,N-3″-oxa-1″,5″-pentamethyleneformamidino)-daunorubicin hydrochloride;
3′-deamino-3′-(N,N-3″-oxa-1″,5″-pentamethyleneformamidino)-daunorubicin;
3′-deamino-3′-(N,N-1″,5″-pentamethyleneformamidino)-daunorubicin hydrochloride;
3′-deamino-3′-(N,N-1″,5″-pentamethyleneformamidino)-daunorubicin;
3′-deamino-3′-(N,N-1″,6″-hexamethyleneformamidino)-daunorubicin hydrochloride;
3′-deamino-3′-(N,N-3″-methylaza-1″,5″-pentamethyleneformamidino)-daunorubicin hydrochloride;
3′-deamino-3′-(N,N-3″-methylaza-1″,5″-pentamethyleneformamidino)-daunorubicin;
3′-deamino-3′-(N,N-o-ethylenephenyleneformamidino)-daunorubicin hydrochloride;
3′-deamino-3′-(N,N-o-ethylenephenyleneformamidino)-daunorubicin;
3′-deamino-3′-(N,N-diethylformamidino)-daunorubicin hydrochloride;
3′-deamino-3′-(N,N-diethylformamidino)-daunorubicin;
3′-deamino-3′-(N,N-dipropylformamidino)-daunorubicin hydrochloride;
3′-deamino-3′-(N,N-dipropylformamidino)-daunorubicin;
3′-deamino-3′-(N,N-dibutylformamidino)-daunorubicin hydrochloride;
3′-deamino-3′-(N,N-dibutylformamidino)-daunorubicin;
3′-deamino-3′-(N,N-1″,4″-tetramethyleneacetamidino)-daunorubicin hydrochloride;
3′-deamino-3′-(N,N-1″,4″-tetramethyleneacetamidino)-daunorubicin;
3′-deamino-3′-(N,N-3″-oxa-1″,5″-pentamethyleneacetamidino)-daunorubicin hydrochloride;
3′-deamino-3′-(N,N-3″-oxa-1″,5″-pentamethyleneacetamidino)-daunorubicin;
3′-deamino-3′-(N,N-1″,5″-pentamethyleneacetamidino)-daunorubicin hydrochloride;
3′-deamino-3′-(N,N-1″,5″-pentamethyleneacetamidino)-daunorubicin;
3′-deamino-3′-(N,N-1″, 6″-hexamethyleneacetamidino)-daunorubicin hydrochloride;
3′-deamino-3′-(N,N-3″-methylaza-1″,5″-pentamethyleneacetamidino)-daunorubicin hydrochloride;
3′-deamino-3′-(N,N-3″-methylaza-1″,5″-pentamethyleneacetamidino)-daunorubicin;
3′-deamino-3′-(N,N-o-ethylenephenyleneacetamidino)-daunorubicin hydrochloride;
3′-deamino-3′-(N,N-o-ethylenephenylenacetamidino)-daunorubicin;
3′-deamino-3′-(N,N-diethylacetamidino)-daunorubicin hydrochloride;
3′-deamino-3′-(N,N-diethylacetamidino)-daunorubicin;
3′-deamino-3′-(N,N-dipropylacetamidino)-daunorubicin hydrochloride;
3′-deamino-3′-(N,N-dipropylacetamidino)-daunorubicin;
3′-deamino-3′-(N,N-dibutylacetamidino)-daunorubicin hydrochloride;
3′-deamino-3′-(N,N-dibutylacetamidino)-daunorubicin;
3′-deamino-3′-(N,N-1″,4″-tetramethyleneformamidino)-doxorubicin hydrochloride;
3′-deamino-3′-(N,N-1″,4″-tetramethyleneformamidino)-doxorubicin;
3′-deamino-3′-(N,N-3″-oxa-1″,5″-pentamethyleneformamidino)-doxorubicin hydrochloride;
3′-deamino-3′-(N,N-3″-oxa-1″,5″-pentamethyleneformamidino)-daunorubicin;
3′-deamino-3′-(N,N-1″,5″-pentamethyleneformamidino)-doxorubicin hydrochloride;
3′-deamino-3′-(N,N-1″,5″-pentamethyleneformamidino)-doxorubicin;
3′-deamino-3′-(N,N-1″,6″-hexamethyleneformamidino)-doxorubicin hydrochloride;
3′-deamino-3′-(N,N-3″-methylaza-1″,5″-pentamethyleneformamidino)-doxorubicin hydrochloride;
3′-deamino-3′-(N,N-3″-methylaza-1″,5″-pentamethyleneformamidino)-doxorubicin;
3′-deamino-3′-(N,N-o-ethylenephenyleneformamidino)-doxorubicin hydrochloride;
3′-deamino-3′-(N,N-o-ethylenephenyleneformamidino)-doxorubicin;
3′-deamino-3′-(N,N-diethylformamidino)-doxorubicin hydrochloride;
3′-deamino-3′-(N,N-diethylformamidino)-doxorubicin;
3′-deamino-3′-(N,N-dipropylformamidino)-doxorubicin hydrochloride;
3′-deamino-3′-(N,N-dipropylformamidino)-doxorubicin;
3′-deamino-3′-(N,N-dibutylformamidino)-doxorubicin hydrochloride;
3′-deamino-3′-(N,N-dibutylformamidino)-doxorubicin;
3′-deamino-3′-(N,N-1″,4″-tetramethyleneacetamidino)-doxorubicin hydrochloride;
3′-deamino-3′-(N,N-1″,4″-tetramethyleneacetamidino)-doxorubicin;
3′-deamino-3′-(N,N-3″-oxa-1″,5″-pentamethyleneacetamidino)-doxorubicin hydrochloride;
3′-deamino-3′-(N,N-3″-oxa-1″,5″-pentamethyleneacetamidino)-doxorubicin;
3′-deamino-3′-(N,N-1″,5″-pentamethyleneacetamidino)-doxorubicin hydrochloride;
3′-deamino-3′-(N,N-1″,5″-pentamethyleneacetamidino)-doxorubicin;
3′-deamino-3′-(N,N-1″, 6″-hexamethyleneacetamidino)-doxorubicin hydrochloride;
3′-deamino-3′-(N,N-3″-methylaza-1″,5″-pentamethyleneacetamidino)-doxorubicin hydrochloride;
3′-deamino-3′-(N,N-3″-methylaza-1″,5″-pentamethyleneacetamidino)-doxorubicin;
3′-deamino-3′-(N,N-o-ethylenephenyleneacetamidino)-doxorubicin hydrochloride;
3′-deamino-3′-(N,N-o-ethylenephenyleneacetamidino)-doxorubicin;
3′-deamino-3′-(N,N-diethylacetamidino)-doxorubicin hydrochloride;
3′-deamino-3′-(N,N-diethylacetamidino)-doxorubicin;
3′-deamino-3′-(N,N-dipropylacetamidino)-doxorubicin hydrochloride;
3′-deamino-3′-(N,N-dipropylformamidino)-doxorubicin;
3′-deamino-3′-(N,N-dibutylacetamidino)-doxorubicin hydrochloride;
3′-deamino-3′-(N,N-dibutylacetamidino)-doxorubicin;
3′-deamino-3′-(N,N-1″,4″-tetramethyleneformamidino)-epidaunorubicin hydrochloride;
3′-deamino-3′-(N,N-1″,4″-tetramethyleneformamidino)-epidaunorubicin;
3′-deamino-3′-(N,N-3″-oxa-1″,5″-pentamethyleneformamidino)-epidaunorubicin hydrochloride;
3′-deamino-3′-(N,N-3″-oxa-1″,5″-pentamethyleneformamidino)-epidaunorubicin;
3′-deamino-3′-(N,N-1″,5″-pentamethyleneformamidino)-epidaunorubicin hydrochloride;
3′-deamino-3′-(N,N-1″,5″-pentamethyleneformamidino)-epidaunorubicin;
3′-deamino-3′-(N,N-1″,6″-hexamethyleneformamidino)-epidaunorubicin hydrochloride;
3′-deamino-3′-(N,N-3″-methylaza-1″,5″-pentamethyleneformamidino)-epidaunorubicin hydrochloride;
3′-deamino-3′-(N,N-3″-methylaza-1″,5″-pentamethyleneformamidino)-epidaunorubicin;
3′-deamino-3′-(N,N-o-ethylenephenyleneformamidino)-epidaunorubicin hydrochloride;
3′-deamino-3′-(N,N-o-ethylenephenyleneformamidino)-epidaunorubicin;
3′-deamino-3′-(N,N-diethylformamidino)-epidaunorubicin hydrochloride;
3′-deamino-3′-(N,N-diethylformamidino)-epidaunorubicin;
3′-deamino-3′-(N,N-dipropylformamidino)-epidaunorubicin hydrochloride;
3′-deamino-3′-(N,N-dipropylformamidino)-epidaunorubicin;
3′-deamino-3′-(N,N-dibutylformamidino)-epidaunorubicin hydrochloride;
3′-deamino-3′-(N,N-dibutylformamidino)-epidaunorubicin;
3′-deamino-3′-[N-methyl-N-(1″-phenylethyl)-formamidino]-epidaunorubicin hydrochloride;
3′-deamino-3′-(N,N-1″,4″-tetramethyleneacetamidino)-epidaunorubicin hydrochloride;
3′-deamino-3′-(N,N-1″,4″-tetramethyleneacetamidino)-epidaunorubicin;
3′-deamino-3′-(N,N-3″-oxa-1″,5″-pentamethyleneacetamidino)-epidaunorubicin hydrochloride;
3′-deamino-3′-(N,N-3″-oxa-1″,5″-pentamethyleneacetamidino)-epidaunorubicin;
3′-deamino-3′-(N,N-1″,5″-pentamethyleneacetamidino)-epidaunorubicin hydrochloride;
3′-deamino-3′-(N,N-1″,5″-pentamethyleneacetamidino)-epidaunorubicin;
3′-deamino-3′-(N,N-1″,6″-hexamethyleneacetamidino)-epidaunorubicin hydrochloride;
3′-deamino-3′-(N,N-3″-methylaza-1″,5″-pentamethyleneacetamidino)-epidaunorubicin hydrochloride;
3′-deamino-3′-(N,N-3″-methylaza-1″,5″-pentamethyleneacetamidino)-epidaunorubicin;
3′-deamino-3′-(N,N-o-ethylenephenyleneacetamidino)-epidaunorubicin hydrochloride;
3′-deamino-3′-(N,N-o-ethylenephenyleneacetamidino)-epidaunorubicin;
3′-deamino-3′-(N,N-diethylacetamidino)-epidaunorubicin hydrochloride;
3′-deamino-3′-(N,N-diethylacetamidino)-epidaunorubicin;
3′-deamino-3′-(N,N-dipropylacetamidino)-epidaunorubicin hydrochloride;
3′-deamino-3′-(N,N-dipropylacetamidino)-epidaunorubicine;
3′-deamino-3′-(N,N-dibutylacetamidino)-epidaunorubicin hydrochloride;
3′-deamino-3′-(N,N-dibutylacetamidino)-epidaunorubicin;
3′-deamino-3′-[N-methyl-N-(1″-phenylethyl)-acetamidino]-epidaunorubicin hydrochloride;
3′-deamino-3′-(N,N-1″,4″-tetramethyleneformamidino)-epidoxorubicin hydrochloride;
3′-deamino-3′-(N,N-1″,4″-tetramethyleneformamidino)-epidoxorubicin;
3′-deamino-3′-(N,N-3″-oxa-1″,5″-pentamethyleneformamidino)-epidoxorubicin hydrochloride;
3′-deamino-3′-(N,N-3″-oxa-1″,5″-pentamethyleneformamidino)-epidoxorubicin;
3′-deamino-3′-(N,N-1″,5″-pentamethyleneformamidino)-epidoxorubicin hydrochloride;
3′-deamino-3′-(N,N-1″,5″-pentamethyleneformamidino)-epidoxorubicin;
3′-deamino-3′-(N,N-1″,6″-hexamethyleneformamidino)-epidoxorubicin hydrochloride;
3′-deamino-3′-(N,N-3″-methylaza-1″,5″-pentamethyleneformamidino)-epidoxorubicin hydrochloride;
3′-deamino-3′-(N,N-3″-methylaza-1″,5″-pentamethyleneformamidino)-epidoxorubicin;
3′-deamino-3′-(N,N-o-ethylenephenyleneformamidino)-epidoxorubicin hydrochloride;
3′-deamino-3′-(N,N-o-ethylenephenyleneformamidino)-epidoxorubicin;
3′-deamino-3′-(N,N-diethylformamidino)-epidoxorubicin hydrochloride;
3′-deamino-3′-(N,N-diethylformamidino)-epidoxorubicin;
3′-deamino-3′-(N,N-dipropylacetamidino)-epidoxorubicin hydrochloride;
3′-deamino-3′-(N,N-dipropylacetamidino)-epidoxorubicin;
3′-deamino-3′-(N,N-dibutylacetamidino)-epidoxorubicin hydrochloride;
3′-deamino-3′-(N,N-dibutylacetamidino)-epidoxorubicin;
3′-deamino-3′-[N-methyl-N-(1″-phenylethyl)-formamidino]-epidoxorubicin hydrochloride;
3′-deamino-3′-(N,N-1″,4″-tetramethyleneacetamidino)-epidoxorubicin hydrochloride;
3′-deamino-3′-(N,N-1″,4″-tetramethyleneacetamidino)-epidoxorubicin;
3′-deamino-3′-(N,N-3″-oxa-1″,5″-pentamethyleneacetamidino)-epidoxorubicin hydrochloride;
3′-deamino-3′-(N,N-3″-oxa-1″,5″-pentamethyleneacetamidino)-epidoxorubicin;
3′-deamino-3′-(N,N-1″,5″-pentamethyleneacetamidino)-epidoxorubicin hydrochloride;
3′-deamino-3′-(N,N-1″,5″-pentamethyleneacetamidino)-epidoxorubicin;
3′-deamino-3′-(N,N-1″,6″-hexamethyleneacetamidino)-epidoxorubicin hydrochloride;
3′-deamino-3′-(N,N-1″,7″-heptamethyleneacetamidino)-epidoxorubicin hydrochloride;
3′-deamino-3′-(N,N-3″-methylaza-1″,5″-pentamethyleneacetamidino)-epidoxorubicin hydrochloride;
3′-deamino-3′-(N,N-3″-methylaza-1″,5″-pentamethyleneacetamidino)-epidoxorubicin;
3′-deamino-3′-(N,N-o-ethylenephenyleneacetamidino)-epidoxorubicin hydrochloride;
3′-deamino-3′-(N,N-o-ethylenephenyleneacetamidino)-epidoxorubicin;
3′-deamino-3′-(N,N-diethylacetamidino)-epidoxorubicin hydrochloride;
3′-deamino-3′-(N,N-diethylacetamidino)-epidoxorubicin;
3′-deamino-3′-(N,N-dipropylacetamidino)-epidoxorubicin hydrochloride;
3′-deamino-3′-(N,N-dipropylacetamidino)-epidoxorubicin;
3′-deamino-3′-(N,N-dibutylacetamidino)-epidoxorubicin hydrochloride;
3′-deamino-3′-(N,N-dibutylacetamidino)-epidoxorubicin;
3′-deamino-3′-[N-methyl-N-(1″-phenylethyl)-acetamidino]-epidoxorubicin hydrochloride;
3′-deamino-3′-N,4′-O-methylidenedaunorubicin;
3′-deamino-3′-N,4′-O-methylidenedoxorubicin;
3′-deamino-3′-N,4′-O-ethylidenedaunorubicin; or
3′-deamino-3′-N,4′-O-ethylidenedoxorubicin

In another embodiment of the invention, provided is a pharmaceutical composition exhibiting an antihelicase activity comprising a pharmaceutically-acceptable carrier, diluent or excipient, and a compound of Formula 1 or Formula 2.

In a class of any of these embodiments, the pharmaceutical composition further comprises a second compound of Formula 1 or Formula 2.

In a class of any of these embodiments, the pharmaceutical composition further comprises another anti-viral drug.

### BRIEF DESCRIPTION OF THE DRAWINGS

Embodiments of the invention will now be described, by example only, and with reference to the following figures, wherein:

FIGS. 1 and 2 show IC₅₀±SD values of exemplary anthracycline antibiotics used in the methods of the invention (solid bars) as compared to IC₅₀±SD values of epidoxorubicin, doxorubicin, epidaunorubicin, and daunorubicin (open bars). FIG. 1 shows compounds having IC₅₀ values of ≦1.0 μM and FIG. 2 shows compounds having IC₅₀ values of ≧1.0 μM.

### DETAILED DESCRIPTION OF REPRESENTATIVE EMBODIMENTS

The studies were carried out using a method described in Boguszewska-Chachulska A. M. et al., FEBS Lett., 567, 253-238, 2004. Advantages of the method as compared to up-to-now used isotope methods (Kim D. W. et al., Biochemistry Biophysics Research Communications 215, 160-166, 1995; and Borowski P. et al, European J. Biochemistry 270, 1645-1653, 1999) are the following: safety, repeatability and possibility to test large amount of samples, e.g. of various inhibitors of helicase at various concentrations, in a number of repetitions, in standard conditions and during a very short time. A reaction takes place on a 96- or 384-well microtitration plate and a course of the reaction is observed in real time.

The above-described method was used to study antihelicase activity of the compounds of the Formula 1 and 2, wherein R¹, R², R³, R⁴, R⁵, R⁶ and X have the meaning defined above, and—for comparison—of the activity of daunorubicin, doxorubicin, epidaunorubicin and epidoxorubicin. The results obtained using the above-mentioned method indicate, that the studied compounds of Formula 1, wherein R¹, R², R³, R⁴, R⁵, R⁶ and X have the above-given meaning, exhibited potent antihelicase action (the IC₅₀ values in the range of 0.03-11.32 μM) and a distinct dependence of the activity on the structure of the studied amidino derivatives. It appeared that the most active acetamidino derivatives had IC₅₀ values of which were in the range of 0.03-0.99 μM. In the case of formamidino derivatives the most active (the IC₅₀ values in the range of 0.36-1.84 μM) were derivatives having pyrrolidine moiety in the amidino group (Table 1 and 2), whereas the least active (the IC₅₀ values ranging from 9.26 to ≧10 μM) were derivatives of Formula 2, wherein R¹, R and R⁴ have the above-given meaning, containing modified daunosamine moiety (compounds 142-145).

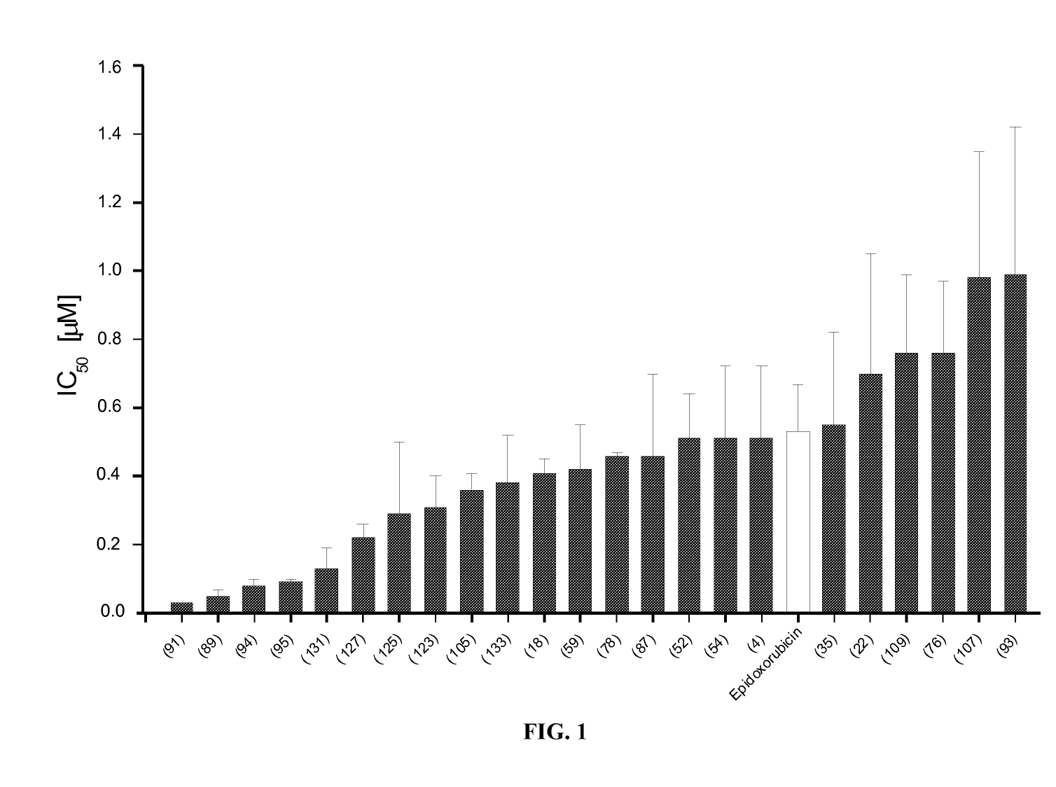

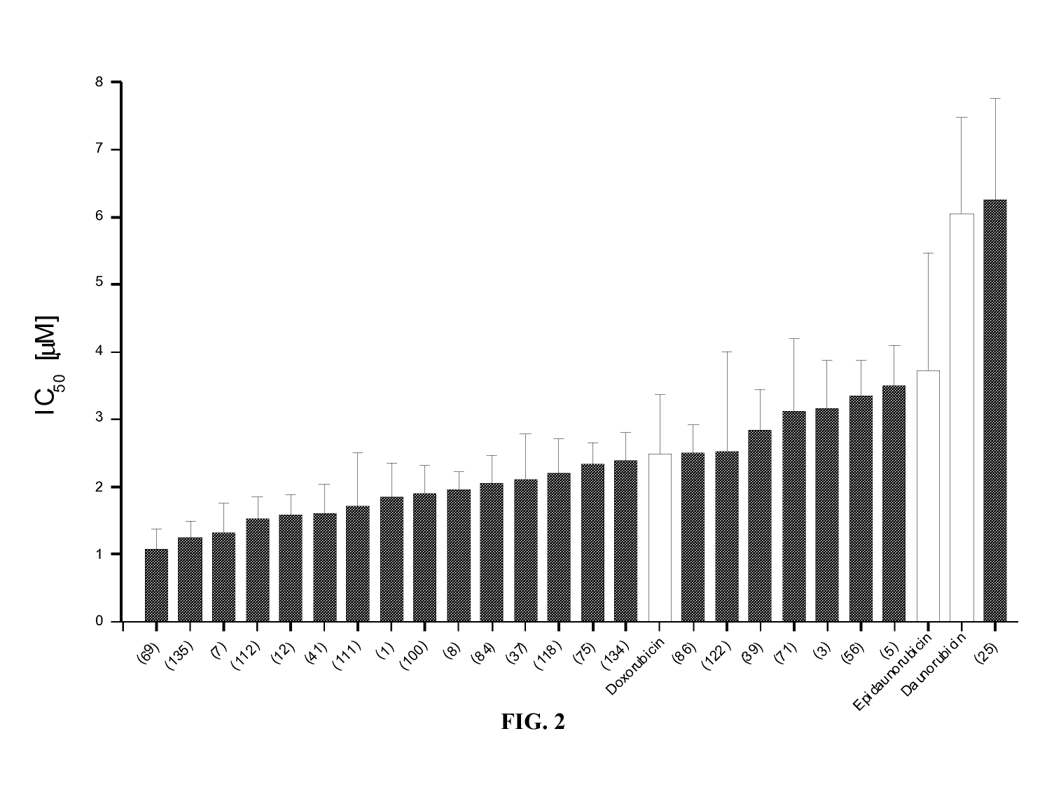

Most of the IC₅₀ values listed in Table 1 and 2 and illustrated by FIGS. 1 and 2 pertain to hydrochlorides of derivatives of the Formula 1, wherein R¹, R², R³, R⁴, R⁵, R⁶ have the meaning defined above, and X is HCl. Studies of activity of these compounds as free bases (if X is 0) exhibited slight differences in the IC₅₀ values between hydrochloride and the corresponding free base, e.g., for hydrochloride of 3′-deamino-3′-(N,N-3″-methylaza-1″,5″-pentamethyleneformamidino)-epidaunorubicin (76) and for corresponding free base-3′-deamino-3′-(N,N-3″-methylaza-1″,5″-pentamethyleneformamidino)-epidaunorubicin (77) these values were: 0.76±0.21 μM and 0.78±0.18 μM, respectively, whereas for hydrochloride of 3′-deamino-3′-(N,N-3″-methylaza-1″,5″-pentamethyleneacetamidino)-epidaunorubi-cin(94) and the free base-3′-deamino-3′-(N,N-3″-methylaza-1″,5″-pentamethylene-acetamidino)-epidaunorubicin (95) were 0.08±0.02 and 0.09±0.01 μM, respectively. Because of that it was assumed that the IC₅₀ values for free bases correspond to the IC₅₀ values for hydrochlorides of compounds of Formula 1 and Formula 2, within IC₅₀±10%.

Therefore the IC₅₀ values for formamidinoanthracyclines as free bases, hydrochlorides of which are listed in Table 1, such as: 3′-deamino-3′-(N,N-1″,4″-tetramethyleneformamidino)-doxorubicin (36), 3′-deamino-3′-(N,N-3″-methylaza-1″,5″-pentamethyleneformamidino)-doxorubicin (43), 3′-deamino-3′-(N,N-3″-methyl-aza-1″,5″-pentamethyleneformamidino)-epidaunorubicin (77), 3′-deamino-3′-(N,N-o-ethylenephenyleneformamidino)-epidaunorubicin (79), 3′-deamino-3′-(N,N-diethyl-formamidino)-epidaunorubicin (81), 3′-deamino-3′-(N,N-1″,4″-tetramethylene-formamidino)-epidoxorubicin(106), 3′-deamino-3′-(N,N-3″-oxa-1″,5″-pentamethylene-formamidino)-epidoxorubicin (108), 3′-deamino-3′-(N,N-1″,5″-pentamethylene-formamidino)-epidoxorubicin (110) and 3′-deamino-3′-(N,N-o-ethylenephenylene-formamidino)-epidoxorubicin (115) are within the limits of 0.4-1.2 μM, whereas analogolously for acetamidinoanthracyclines as free bases (Table 1), such as: 3′-deamino-3′-(N,N-1″,4″-tetramethyleneacetamidino)-daunorubicin (19), 3′-deamino-3′-(N,N-1″, 5″-pentamethyleneacetamidino)-daunorubicin (23), 3′-deamino-3′-(N,N-1″,4″-tetramethyleneacetamidino)-doxorubicin (53), 3′-deamino-3′-(N,N-3″-oxa-1″,5″-pentamethyleneacetamidino)-doxorubicin (55), 3′-deamino-3′-(N,N-3″-methylaza-1″,5″-pentamethyleneacetamidino)-doxorubicin (60), 3′-deamino-3′-(N,N-1″,4″-tetra-methyleneacetamidino)-epidaunorubicin (88), 3′-deamino-3′-(N,N-3″-oxa-1″,5″-penta-methyleneacetamidino)-epidaunorubicin (90), 3′-deamino-3′-(N,N-1″,5″-penta-methyleneacetamidino)-epidaunorubicin (92), 3′-deamino-3′-(N,N-diethyl-acetamidino)-epidaunorubicin (99), 3′-deamino-3′-(N,N-1″,4″-tetramethylene-acetamidino)-epidoxorubicin (124), 3′-deamino-3′-(N,N-3″-oxa-1″,5″-penta-methyleneacetamidino)-epidoxorubicin (126), 3′-deamino-3′-(N,N-1″,5″-penta-methyleneacetamidino)-epidoxorubicin (128), 3′-deamino-3′-(N,N-3″-methylaza-11″,5″-pentamethyleneacetamidino)-epidoxorubicin (132) and 3′-deamino-3′-(N,N-o-ethylenephenyleneacetamidino)-epidoxorubicin (134), within the range 0.08-1.1 μM, respectively.

The antihelicase activity (≧1.0 μM) of derivatives of Formula 1, wherein R¹, R², R³, R⁴, R¹, R⁶ and X have the meaning defined above, is presented in Table 2. That group of compounds includes mainly formamidinoanthracyclines of activity within the IC₅₀ values ranging from 1.08 to 3.5 μM and few acetamidinoanthracyclines of the IC₅₀ values in the range of 1.24-11.32 μM.

Antihelicase activity of free bases corresponding to hydrochlorides of formamidinoanthracyclines listed in Table 2, such as 3′-deamino-3′-(N,N-1″,4″-tetramethyleneformamidino)-daunorubicin (2), 3′-deamino-3′-(N,N-3″-oxa-1″, 5″-pentamethyleneformamidino)-daunorubicin (4), 3′-deamino-3′-(N,N-1″,5″-penta-methyleneformamidino)-daunorubicin (6), 3′-deamino-3′-(N,N-3″-methylaza-1″,5″-pentamethyleneformamidino)-daunorubicin (9), 3′-deamino-3′-(N,N-diethyl-formamidino)-daunorubicin (13), 3′-deamino-3′-(N,N-3″-oxa-1″,5″-pentamethylene-formamidino)-doxorubicin (38), 3′-deamino-3′-(N,N-1″,5″-pentamethylene-formamidino)-doxorubicin (40), 3′-deamino-3′-(N,N-diethylformamidino)-doxorubicin (47), 3′-deamino-3′-(N,N-1″,4″-tetramethyleneformamidino)-epidaunorubicin (70), 3′-deamino-3′-(N,N-3″-oxa-1″,5″-pentamethyleneformamidino)-epidaunorubicin (72), 3′-deamino-3′-(N,N-1″,5″-pentamethyleneformamidino)-epidaunorubicin (74), 3′-deamino-3′-(N,N-dibutylformamidino)-epidaunorubicin (85), 3′-deamino-3′-(N,N-3″-methylaza-1″,5″-pentamethyleneformamidino)-epidoxorubicin (113) 3′-deamino-3′-(N,N-diethylformamidino)-epidoxorubicin (117) and 3′-deamino-3′-(N,N-dipropyl-formamidino)-epidoxorubicin (119), expressed as the IC₅₀ values, were in the range of 1.2-3.9 μM, whereas activity of free bases-acetamidinoanthracyclines (Table 2), such as 3′-deamino-3′-(N,N-3″-methylaza-1″,5″-pentamethyleneacetamidino)-daunorubicin (26), 3′-deamino-3′-(N,N-o-ethylenephenyleneacetamidino)-daunorubicin (28), 3′-deamino-3′-(N,N-1″,5″-pentamethyleneacetamidino)-doxorubicin (57), 3′-deamino-3′-(N,N-dipropylacetamidino)-epidaunorubicin (101) and 3′-deamino-3′-(N,N-diethylacetamidino)-epidoxorubicin (136), expressed as the IC₅₀ values were in the range of 7.0-13.0 μM.

It is important that compounds not mentioned above of Formula 1, wherein R¹, R², R⁵, R⁴, R⁵, R⁶ and X have the meaning defined above, such as 3′-deamino-3′-(N,N-o-ethylenephenyleneformamidino)-daunorubicin hydrochloride (10), 3′-deamino-3′-(N,N-o-ethylenephenyleneformamidino)-daunorubicin (11), 3′-deamino-3′-(N,N-dipropylformamidino)-daunorubicin hydrochloride (14), 3′-deamino-3′-(N,N-dipropylformamidino)-daunorubicin (15), 3′-deamino-3′-(N,N-dibutyl-formamidino)-daunorubicin hydrochloride (16), 3′-deamino-3′-(N,N-dibutyl-formamidino)-daunorubicin (17), 3′-deamino-3′-(N,N-3″-oxa-1″,5″-pentamethylene-acetamidino)-daunorubicin hydrochloride (20), 3′-deamino-3′-(N,N-3″-oxa-1″,5″-pentamethyleneacetamidino)-daunorubicin (21), 3′-deamino-3′-(N,N-1″,6″-hexa-methyleneacetamidino)-daunorubicin hydrochloride (24), 3′-deamino-3′-(N,N-diethylacetamidino)-daunorubicin hydrochloride (29), 3′-deamino-3′-(N,N-diethylacetamidino)-daunorubicin (30), 3′-deamino-3′-(N,N-dipropylacetamidino)-daunorubicin hydrochloride (31), 3′-deamino-3′-(N,N-dipropylacetamidino)-daunorubicin (32), 3′-deamino-3′-(N,N-dibutylacetamidino)-daunorubicin hydrochloride (33), 3′-deamino-3′-(N,N-dibutylacetamidino)-daunorubicin (34), 3′-deamino-3′-(N,N-o-ethylenephenyleneformamidino)-doxorubicin hydrochloride (44), 3′-deamino-3′-(N,N-o-ethylenephenyleneformamidino)-doxorubicin (45), 3′-deamino-3′-(N,N-dipropylformamidino)-doxorubicin hydrochloride (48), 3′-deamino-3′-(N,N-dipropylformamidino)-doxorubicin (49), 3′-deamino-3′-(N,N-dibutylformamidino)-doxorubicin hydrochloride (50), 3′-deamino-3′-(N,N-dibutylformamidino)-doxorubicin (51),3′-deamino-3′-(N,N-1″,6″-hexamethyleneacetamidino)-doxorubicin hydrochloride (58), 3′-deamino-3′-(N,N-o-ethylenephenyleneacetamidino)-doxorubicin hydrochloride(61),3′-deamino-3′-(N,N-o-ethylenephenyleneacetamidino)-doxorubicin (62), 3′-deamino-3′-(N,N-diethylacetamidino)-doxorubicin hydrochloride (63), 3′-deamino-3′-(N,N-diethylacetamidino)-doxorubicin (64), 3′-deamino-3′-(N,N-dipropyl-acetamidino)-doxorubicin hydrochloride (65), 3′-deamino-3′-(N,N-dipropyl-acetamidino)-doxorubicin (66), 3′-deamino-3′-(N,N-dibutylacetamidino)-doxorubicin hydrochloride (67), 3′-deamino-3′-(N,N-dibutylacetamidino)-doxorubicin (68), 3′-deamino-3′-(N,N-dipropylformamidino)-epidaunorubicin hydrochloride (82), 3′-deamino-3′-(N,N-dipropylformamidino)-epidaunorubicin (83), 3′-deamino-3′-(N,N-o-ethylenephenyleneacetamidino)-epidaunorubicin hydrochloride (96), 3′-deamino-3′-(N,N-o-ethylenephenyleneacetamidino)-epidaunorubicin (97), 3′-deamino-3′-(N,N-dibutylacetamidino)-epidaunorubicin hydrochloride (102), 3′-deamino-3′-(N,N-dibutylacetamidino)-epidaunorubicin (103), 3′-deamino-3′-[N-methyl-N-(1″-phenyl-ethyl)-acetamidino]-epidaunorubicin hydrochloride (104), 3′-deamino-3′-(N,N-dibutyl-formamidino)-epidoxorubicin hydrochloride (120), 3′-deamino-3′-(N,N-dibutyl-formamidino)-epidoxorubicin (121), 3′-deamino-3′-(N,N-1″,6″-hexamethylene-acetamidino)-epidoxorubicin hydrochloride (129), 3′-deamino-3′-(N,N-1″,7″-hepta-methyleneacetamidino)-epidoxorubicin hydrochloride (130), 3′-deamino-3′-(N,N-dipropylacetamidino)-epidoxorubicin hydrochloride (137), 3′-deamino-3′-(N,N-dipropylacetamidino)-epidoxorubicin (138), 3′-deamino-3′-(N,N-dibutylacetamidino)-epidoxorubicin hydrochloride (139), 3′-deamino-3′-(N,N-dibutylacetamidino)-epidoxorubicin (140) and 3′-deamino-3′-[N-methyl-N-(1″-phenylethyl)-acetamidino]-epidoxorubicin hydrochloride (141) also exhibit potent antihelicase action. The IC₅₀ values of the above-mentioned derivatives are in the range of between 0.1 and 110.0 M.

It should be pointed out that the most potent antihelicase action exhibited these amidinoanthracyclines which in the group of investigated derivatives of the Formula 1 or Formula 2, wherein R¹, R², R³, R⁴, R⁵, R⁶ and X have the meaning defined above, had the weakest antineoplastic action, which, as it is known, is advantageous when studying antiviral action of individual compounds.

Idarubicine, an orally administered analogue of daunorubicin, having hydrogen in position 3 and its amidinoderivatives—prepared by the Inventors—as 3′-deamino-3′-(N,N-3″-oxa-1″,5″-pentamethyleneformamidino)-idarubicin hydrochloride of the Formula 1, wherein R¹, R² and R⁴ are hydrogen, R³ is hydroxy group in an axial orientation, whereas R⁵ and R⁶ together with a nitrogen atom are N,N-3″-oxa-1″,5″-pentamethylene group and 3′-deamino-3′-(N,N-1″,6″-hexamethyleneformamidino)-idarubicin hydrochloride of Formula 1, wherein R¹, R² and R⁴ are hydrogen, R³ is hydroxy group in an axial orientation, whereas R⁵ and R⁶ together with a nitrogen atom form N,N-1″,6″-hexamethylene group, also exhibited an antihelicase action, but the action was less potent (≧10 μM) than that of epidoxorubicin, doxorubicin, epidaunorubicin, or daunorubicin, as well as that of their derivatives of Formula 1.

As it has been found in several studies, an antihelicase activity of compounds is mainly associated with their antiviral activity. This results from the crucial role of NS3 protein in the replication cycle of Flaviviridae viruses, which because of that can be a target of antiviral chemotherapy (Tan S-L at al., Nature Reviews/Drug Discovery 1, 867-881, 2002, Gordon C. P. and Keller P. A., J. Med. Chem. 48, 1-20, 2005). For example, a study concerning inhibition of multiplication of a virus of the Flaviviridae family (Western Nile Virus, WNV) conducted by Borowski and coll. (Borowski P. et al., Antimicrobial Agent and Chemotherapy 46, 1231-1239, 2002, and Borowski P. et al, (Acta. Biochim. Polon. 49, 597-614, 2002) proves that a nucleoside analogue, which is an inhibitor of viral helicase activity under conditions without an incubation with a substrate, at the same time is an effective inhibitor of virus replication in a cell line, which in both cases makes it possible to attain 50% inhibition at similar concentration (approx. 30 μM). Studies of inhibitors of helicase-primase of herpes simplex virus (HSV), using modified nucleosides analogues, also exhibited that these compounds can be effective inhibitors of viral replication, 50% inhibition of replication occurring at lower concentrations than inhibition of helicase activity (Crute J. J. et al., Nature Medicine 8, 386-391, 2002).

The derivatives in question of Formula 1 and Formula 2 are subject to further testing in HCV-infected human lymphocytes and peripheral blood mononuclear cells (PBMC).

Studies on inhibition of HCV replication, using human liver cancer cell line (Huh7) carrying a subgenomic HCV replicon of genotype 1 (con1) with a reporter/selection fusion protein luc-ubi-neo, were also carried out. Results of determination of antiviral activity and therapeutic index of selected amidinoanthracyclines are given in Table 3. It has been found that the derivatives in question reduced level of HCV RNA in a concentration-dependent manner, efficiently inhibiting replication of HCV at nanomolar concentrations (from 8 to 140 nM) without cell cytotoxicity, with exception of an epidaunorubicin derivative (75), which, however, is the most potent inhibitor, presenting activity (EC₅₀=8.4 nM) 4 fold higher than that of epidoxorubicin, doxorubicin, epidaunorubicin, or daunorubicin (EC₅₀=31.18 nM).

Although a doxorubicin derivative (54) has higher EC₅₀ than doxorubicin, it exhibits the lowest cytotoxicity of the studied compounds and the highest therapeutic index. It should be pointed out that all derivatives that were compared exhibited high therapeutic indices (5.7, 8.0, 11.4, 19.2 and 33.3), whereas for ribavirin—a drug used at present in treatment of infection by HCV—the index was merely 1.5, at EC₅₀=120 μM. These data suggest that derivatives (54, 75, 94, 125, 133) can be more preferable components of future multicomponent therapy against HCV than ribavirin.

As pharmaceutically acceptable carriers and diluents preferably lactose, nipagin, dextrose, glucose or 0.9% sodium chloride solution are used.

Compounds of Formula 1 or Formula 2 can be administered as injections or infusions. For the purpose of parenteral administration, unit dosage forms are prepared as solutions that include each of the compounds according to the invention and an aseptic diluent. When preparing the solution, the active ingredient can be dissolved in a solution for injections and sterilized by filtering. The resulting sterile solution is used to fill vials or ampoules, which are then closed under aseptic conditions. The solution in vials can also be lyophilized to obtain Formulation as a dry powder. In such cases a second vial is added to a lyophilizate including water or solution for injections, to prepare an injection drug form. The above-mentioned drug forms as an active ingredient can also include free bases or hydrochlorides of each of the compounds according to the invention.

Accordingly, compounds of Formula 1 or Formula 2, wherein R¹, R², R³, R⁴, R⁵, R⁶ and X have the above-given meaning, exhibit potent antihelicase action and -previously disclosed-considerably lower toxicity as compared to epidoxorubicin, doxorubicin, epidaunorubicin, or daunorubicin, and can be preferably used for treatment of HCV infections in the future.

Embodiments of the invention will now be described in greater detail with reference to the following examples which are given here for illustrative purposes only and are by no means intended to limit the scope of the invention.

### Example 1

### Antihelicase Activity

The helicase domain of NS3 protein was expressed in a baculovirus system in HF insect cell line. The helicase protein obtained in the baculovirus system was purified using a method described in Boguszewska-Chachulska A. M. et al., FEBS Lett. 567: 253-258, 2004, consisting of following steps:
Stage I—chromatographic separation of insect cells lysate on a Talon column (Clontech), using affinity of cobalt ions to histidine repetitions, and elution of the protein from the column with 250 mM imidazole;
Stage II—further purification of the protein by affinity method on a heparin column HiTrap (Amersham Biosciences) using Biologic (BioRad) or AKTA (Amersham Biosciences) system, optionally followed by concentration of the eluate with Amicon Ultra filters (Millipore).

Antihelicase activity was examined using a fluorometric assay based on the use of a double-stranded DNA substrate, where one strand was labeled at the 5′ end with a fluorophore (FAM) and the second strand was labeled at 3′ end with Black Hole Quencher (BHQ1), a molecule absorbing fluorescence (Boguszewska-Chachulska A. M. et al., FEBS Lett. 567: 253-258, 2004). In this assay, FAM fluorescence is quenched by fluorescence energy transfer (FRET) to the BHQ molecule, while unwinding of double-stranded DNA structure leads to decrease of FRET phenomenon and increase of FAM fluorescence signal. The assay allows for examining a large number of samples, i.e., different helicase inhibitors at various concentrations and with many repetitions, in standardized conditions. The reaction is conducted in a microtitration plate. The reaction process is observed in real time.

The examination of potential inhibitors of HCV helicase was conducted under conditions described in Boguszewska-Chachulska A. M. et al. (Biochem Biophys Res Commun. 34: 641-647, 2006). Helicase reactions were performed in the following mixture: 30 mM Tris-HCl, pH 7.5, 10 mM MnCl₂, 0.075% Triton X-100, 0.05% sodium azide, 10 nM substrate, 1.5 mM ATP, 125 nM of oligonucleotide complementary to BHQ1-labelled strand in the 60 μL reaction volume. The enzyme (10 nM) was pre-incubated with the tested derivatives dissolved in DMSO (0.005 to 20 μM concentration) without ATP for 15 min at room temperature. The reaction of DNA unwinding was started by addition of ATP and was carried out at 30° C. for 60 min in a Synergy HTi (Biotek) fluorometer. Since the excitation maximum for FAM is at 495 nm, and the emission maximum is at 520 nm, the fluorophore was excited using a 485/20 nm filter, and the helicase activity was measured at 528/20 nm. Fluorescence was measured every 2 mins.

The enzyme activity was calculated as the initial reaction velocity from the linear part of reaction curve (fluorescence growth as a function of time) using a linear regression method. Inhibition was calculated as comparison of activity of the helicase incubated with inhibitor to activity of the helicase incubated without inhibitor, but with a proper concentration of DMSO, expressed in percent. Obtained IC₅₀ values in μM (molar concentration of examined compound which inhibits 50% of helicase activity) are listed in Tables 1 and 2 and illustrated in FIGS. 1 and 2.

TABLE 1
IC₅₀ ± SD values of exemplary anthracycline antibiotics used in the
methods of the invention as compared to IC₅₀ ± SD values of
epidoxorubicin, doxorubicin, epidaunorubicin, and daunorubicin (Part 1)*.

| Derivative | IC₅₀ | SD |
|---|---|---|
| (ref. number or name) | (μM) | (μM) |
| (91) | 0.03 | 0.00 |
| (89) | 0.05 | 0.02 |
| (94) | 0.08 | 0.02 |
| (95) | 0.09 | 0.01 |
| (131) | 0.13 | 0.06 |
| (114) | 0.14 | 0.02 |
| (98) | 0.18 | 0.02 |
| (127) | 0.22 | 0.04 |
| (125) | 0.29 | 0.21 |
| (80) | 0.30 | 0.07 |
| (123) | 0.31 | 0.09 |
| (105) | 0.36 | 0.05 |
| (133) | 0.38 | 0.14 |
| (18) | 0.41 | 0.04 |
| (59) | 0.42 | 0.13 |
| (78) | 0.45 | 0.01 |
| (87) | 0.46 | 0.24 |
| (52) | 0.50 | 0.13 |
| (54) | 0.51 | 0.21 |
| Epidoxorubicin | 0.53 | 0.14 |
| (35) | 0.55 | 0.27 |
| (22) | 0.70 | 0.35 |
| (109) | 0.76 | 0.23 |
| (76) | 0.77 | 0.21 |
| (107) | 0.97 | 0.07 |
| (42) | 0.98 | 0.11 |
| (93) | 0.99 | 0.86 |

*sorted by ascending IC₅₀ values

TABLE 2
IC₅₀ ± SD values of exemplary anthracycline antibiotics used in the
methods of the invention as compared to IC₅₀ ± SD values of
epidoxorubicin, doxorubicin, epidaunorubicin, and daunorubicin (Part 2)*.

| Derivative | IC₅₀ | SD |
|---|---|---|
| (ref. number or name) | (μM) | (μM) |
| (69) | 1.08 | 0.30 |
| (135) | 1.24 | 0.26 |
| (46) | 1.32 | 0.07 |
| (7) | 1.33 | 0.43 |
| (112) | 1.53 | 0.31 |
| (12) | 1.58 | 0.30 |
| (41) | 1.61 | 0.42 |
| (111) | 1.72 | 0.78 |
| (1) | 1.84 | 0.52 |
| (100) | 1.90 | 0.42 |
| (8) | 1.96 | 0.26 |
| (84) | 2.05 | 0.41 |
| (37) | 2.12 | 0.66 |
| (116) | 2.17 | 0.45 |
| (118) | 2.21 | 0.51 |
| (75) | 2.33 | 0.33 |
| (73) | 2.39 | 0.41 |
| Doxorubicin | 2.48 | 0.88 |
| (86) | 2.50 | 0.42 |
| (122) | 2.53 | 1.47 |
| (39) | 2.84 | 0.61 |
| (71) | 3.13 | 1.06 |
| (3) | 3.17 | 0.71 |
| (56) | 3.35 | 0.52 |
| (5) | 3.50 | 0.59 |
| Epidaunorubicin | 3.73 | 1.73 |
| Daunorubicin | 6.05 | 1.43 |
| (25) | 6.25 | 1.51 |
| (142) | 9.26 | 2.03 |
| (27) | 11.32 | 0.98 |
| (144) | >6 | — |
| (143) | >10 | — |
| (145) | >10 | — |

*sorted by ascending IC₅₀ values

### Example 2

### Antiviral Activity and Therapeutic Index

In order to examine antiviral activity and to determine therapeutic index of exemplary anthracycline antibiotics, subgenomic replicon of hepatitis C virus was used, which is a modified genome of HCV lacking structural genes. The replicon was a non-infective form of the virus, and therefore is safe to work with and makes it easier for high-throughput screening of potential inhibitors of its replication.

Additional simplification was the use of reporter protein—luciferase, whose activity (i.e., luminescence generated by it after addition of substrate) was proportional to the amount of the protein expressed, which allowed determination of HCV RNA replication level and its inhibition by virus replication inhibitors.

The human hepatoma cell line Huh-7 and the plasmid pFK-luc-ubi-neo/NS3-3′/Con1/5.1 carrying the subgenomic HCV genotype 1 (con 1) replicon with the luc-ubi-neo reporter/selective fusion protein (Vrolijk J. M. et al., J. Virol. Methods 110: 201-209, 2003), used in the experiments, were provided by Dr. Ralf Bartenschlager (Department of Molecular Virology, University of Heidelberg, Heidelberg). Huh-7 clones carrying persistently-replicating subgenomic HCV replicons were obtained using the following modification of the protocol described by Lohman V. et al. (J. Virol. 75:1437-1449, 2001).

Huh-7 cells were grown in minimal essential medium (Dulbecco's Modified Minimal Essential Medium, DMEM; Invitrogen) with high glucose concentration (4.5 g/L) supplemented with 2 mM L-glutamine and 1 μM non-essential amino acids, such as: glycine, L-alanine, L-asparagine, L-aspartic acid, L-glutamic acid, L-proline and L-serine, and also with 100 U/mL penicilin, 100 μg/mL streptomycin, and 10% fetal bovine serum (FBS, Sigma). In cultures of cells bearing the subgenomic HCV replicon 250 μg/mL of aminoglycoside antibiotics G418 (Geneticin; Invitrogen) was added to the medium. Cells were grown at 37° C. (5% CO₂) and were passaged twice per week, diluted 7 to 10 times, according to the protocol described in Vrolijk J. M. et al., J. Virol. Methods 110:201-209, 2003.

The Huh-7 cells carrying replicons were cultivated with the following concentrations of anthracycline antibiotics: 5 to 200 nM for 4 days to determine their influence on HCV RNA replication and 5 to 5000 nM for 3 days to determine cytotoxicity. The medium with 1% DMSO was used as a control.

Conditions of the antiviral activity test of antracycline antibiotics were based on the protocol described by Paeshuyse J. et al. (Biochem. Biophys. Res. Commun. 348: 139-144, 2006). A logarithmic culture of HCV replicon-carrying cells was diluted in complete DMEM medium supplemented with 250 μg/ml G418 to the concentration of 5-7×10³ cells per mL and aliquoted into 96-well white microtitration plate with optical bottom (Nunc) (100 μL per well). After 24 h at 37° C. the medium was removed and dilution of inhibitors in the range of 5 to 200 nM was added in 100 μL of fresh DMEM medium without G418. Huh-7 cells with 1% DMSO were used as a control. After 4 days at 37° C., the medium was removed and 40 μL of mixture of Glo Lysis buffer and Bright-Glo luciferase assay system (Promega) was added to each well. After 2 mins of incubation luminescence was measured with a Synergy HTi (Biotek) device. The experiment was repeated at least three times with three repetitions for each inhibitor concentration. EC₅₀ value (50% effective concentration) was calculated as the concentration of the inhibitor that reduced by 50% the luminescence corresponding to the HCV RNA replication level.

When determining cytotoxicity, as in the antiviral test, logarithmic culture of HCV replicon-carrying cells was diluted in complete DMEM medium supplemented with 250 μg/mL G418 to the concentration of 5−7×10³ cells per mL and aliquoted into 96-well mictotitration cell culture plate (100 μL per well). After 24 h at 37° C. medium was removed and inhibitors were added at concentrations of 5 to 5000 nM in 100 μL of fresh DMEM medium without G418. After 3 days at 37° C., the medium was removed and 100 μL of fresh DMEM medium with 0.5 mg/mL of MTT (tetrazolium bromide, Sigma) was added to each well. After 3 to 4 hours of incubation at 37° C., the medium with MTT was removed and 100 μL of 0.04 N HCl solution in absolute isopropanol was added to solubilize the precipitated dye. Dye absorbance was measured at 570 nm, while absorbance of the background was measured at 650 nm using Synergy HTi (Biotek). The experiment was repeated at least 3 times with 3 repetitions for each inhibitor concentration. After background subtraction CC₅₀ value (50% cytotoxicity) was calculated as the inhibitor concentration that inhibited the cell growth by 50%.

Values of EC₅₀, CC₅₀ and therapeutic index (TI=CC₅₀/EC₅₀) obtained for exemplary anthracycline antibiotics used in the methods of the invention, and for comparison for epidoxorubicin and doxorubicin, are shown in Table 3.

TABLE 3
EC₅₀ ± SD, CC₅₀ ± SD and TI values for exemplary anthracycline
antibiotics used in the methods of the invention, and for
comparison for epidoxorubicin and doxorubicin

| Derivative | EC₅₀ | | CC₅₀ | | |
|---|---|---|---|---|---|
| (ref. number or name) | (nM) | ±SD | (nM) | ±SD | TI |
| 75 | 8.40 | 2.82 | 47.85 | 13.3 | 5.7 |
| 94 | 56.87 | 3.04 | 1092.45 | 168.24 | 19.2 |
| 125 | 114.04 | 47.73 | 909.49 | 213.5 | 8.0 |
| 54 | 128.58 | 32.45 | 4280.63 | 437.7 | 33.3 |
| 133 | 137.64 | 47.42 | 1567.23 | 508.7 | 11.4 |
| Doxorubicin | 42.91 | 8.96 | 332.38 | 72.6 | 7.7 |
| Epidoxorubicin | 73.47 | 12.61 | 674.93 | 107.7 | 9.2 |

### Example 3

### 3′-Deamino-3′-(N,N-3″-methylaza-1″,5″-pentamethyleneacetamidino)-epidoxorubicin hydrochloride (131) for Injections

To 210 mL of apyrogene water 100 mg of 3′-deamino-3′-(N,N-3″-methylaza-1″,5″-pentamethyleneacetamidino)-epidoxorubicin hydrochloride (131), 0.5 g of lactose, 10 mg of nipagin M and 100 mg of sodium chloride were added and mixed until dissolution. Then, the resulting solution was filtered through an aseptic filter and it was dosed 3.5 mL each to 10 mL glass vials and upon freezing to −45° C., it was freeze dried.

56 vials for injections containing: 1.6±0.1 mg of 3′-deamino-3′-(N,N-3″-methylaza-1″,5″-pentamethyleneacetamidino)-epidoxorubicin hydrochloride (131), 8.3±0.2 mg of lactose, 0.16±0.01 mg of nipagin M, and 1.6±0.1 mg of sodium chloride, were obtained.

### Example 4

3′-Deamino-3′-(N,N-1″,5″-penta-methyleneacetamidino)-epidaunorubicin hydrochloride (91) for Injections

To 210 mL of water, 60 mg of 3′-deamino-3′-(N,N-1″,5″-penta-methyleneacetamidino)-epidaunorubicin hydrochloride (91), 0.6 g of lactose, 12 mg of nipagin M, and 100 mg of sodium chloride were added and mixed until dissolution. Then, the resulting solution was filtered through an aseptic filter and dosed 3.5 mL each to 10 mL glass vials and upon freezing to −45° C., it was freeze dried. 56 vials for injections containing: 1.0±0.1 mg of 3′-deamino-3′-(N,N-1″,5″-pentamethyleneacetamidino)-epidauno-rubicin (91), 10.0±0.2 mg of lactose, 0.2±0.01 mg of nipagin M, 1.6±0.1 mg of sodium chloride, were obtained.

### Example 5

### Mixture of 3′-Deamino-3′-(N,N-1″,4″-tetramethylene-formamidino)-epidoxorubicin hydrochloride (123) and 3′-Deamino-3′-(N,N-3″-methylaza-1″,5″-pentamethyleneacetamidino)-epidaunorubicin hydrochloride (94) for Injections

To 210 mL of water, 50 mg of 3′-deamino-3′-(N,N-1″,4″-tetramethylene-formamidino)-epidoxorubicin hydrochloride (123), 50 mg of 3′-deamino-3′-(N,N-3″-methylaza-1″,5″-pentamethyleneacetamidino)-epidaunorubicin hydrochloride (94), 0.75 g of lactose, 15 mg of nipagin M and 50 mg of sodium chloride were added and mixed until dissolution. Then, the resulting solution was filtered through an aseptic filter, dosed 3.6 mL each to 10 mL glass vials and upon freezing to −45° C. it was freeze dried. 55 vials for injections containing: 0.85±0.05 mg of 3′-deamino-3′-(N,N-1″,4″-tetramethyleneformamidino)-epidoxorubi-cin hydrochloride (123), 0.85±0.05 mg of 3′-deamino-3′-(N,N-3″-methylaza-1″,5″-pentamethylene-acetamidino)-epidaunorubicin hydrochloride (94), 12.8±0.2 mg of lactose, 0.26±0.01 mg of nipagin M, and 0.85±0.05 mg of sodium chloride, were obtained.

### Example 6

### 3′-Deamino-3′-(N,N-1″,5″-pentamethyleneformamidino)-epidaunorubicin hydrochloride (73) for Injections

To 210 mL of water, 50 mg of doxorubicin hydrochloride, 150 mg of 3′-deamino-3′-(N,N-1″,5″-pentamethyleneformamidino)-epidaunorubicin hydrochloride (73), 0.75 g of lactose and 15 mg of nipagin M were added and mixed until dissolution. Then, the resulting solution was filtered through an aseptic filter, dosed 3.6 mL each to 10 mL glass vials and upon freezing to −40° C. it was freeze dried. 57 vials for injections, containing: 0.85±0.05 mg of doxorubicin hydrochloride, 2.5±0.1 mg of 3′-deamino-3′-(N,N-1″,5″-pentamethyleneformamidino)-epidauno-rubicin hydrochloride (73), 12.8±0.2 mg of lactose, and 0.26±0.01 mg of nipagin M, were obtained.

### Example 7

### Mixture of 3′-deamino-3′-(N,N-1″,4″-tetramethyleneformamidino)-doxorubicin hydrochloride (35) and 3′-deamino-3′-(N,N-3″-methylaza-1″,5″-pentamethyleneacetamidino)-epidoxorubicin hydrochloride (131) for Injections

To 230 mL of water, 50 mg of doxorubicin hydrochloride, 100 mg of 3′-deamino-3′-(N,N-1″,4″-tetramethyleneformamidino)-doxorubicin hydrochloride (35) 50 mg of 3′-deamino-3′-(N,N-3″-methylaza-1″,5″-pentamethyleneacetamidino)-epidoxorubicin hydrochloride (131), 0.75 g of lactose and 15 mg of nipagin M were added and mixed until dissolution. Then, the resulting solution was filtered through an aseptic filter, dosed 3.7 mL each to 10 mL glass vials. Upon freezing to −45° C., it was freeze dried. 58 vials containing: 0.8±0.05 mg of doxorubicin hydrochloride, 1.6±0.13 mg of 3′-deamino-3′-(N,N-1″,4″-tetramethyleneformamidino)-doxorubicin hydrochloride (35), 0.8±0.05 mg of 3′-deamino-3′-(N,N-3″-methylaza-1″,5″-pentamethylene-acetamidino)-epidoxorubicin hydrochloride (131), 12.0±0.2 mg of lactose, 0.24±0.01 mg of nipagin M, were obtained.

This invention is not to be limited to the specific embodiments disclosed herein and modifications for various applications and other embodiments are intended to be included within the scope of the appended claims. While this invention has been described in connection with particular examples thereof, the true scope of the invention is not so limited since other modifications will become apparent to the skilled practitioner upon a study of the drawings, specification, and following claims.

All publications and patent applications mentioned in this specification are indicative of the level of skill of those skilled in the art to which this invention pertains. All publications and patent applications mentioned in this specification are herein incorporated by reference to the same extent as if each individual publication or patent application mentioned in this specification was specifically and individually indicated to be incorporated by reference.

The invention claimed is:

1. A method of treating hepatitis C viral infection or viremia in a patient comprising administering to the patient an anti-hepatitis C virally effective amount of:
(a) a compound of Formula 1 or Formula 2, or
(b) a pharmaceutical composition comprising a pharmaceutically-acceptable carrier, diluent or excipient and a compound of Formula 1 or Formula 2, 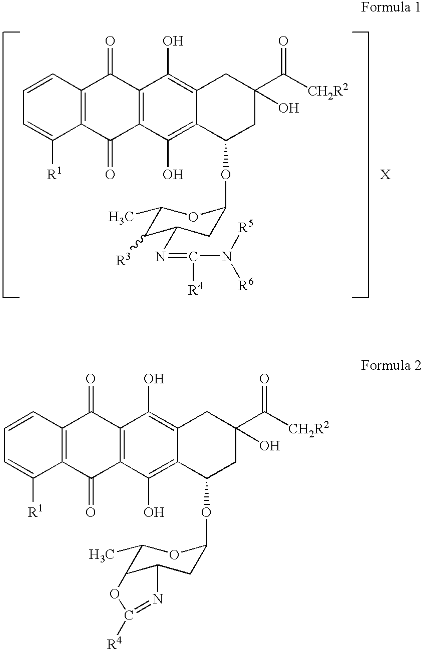
wherein
R¹ is hydrogen or methoxy;
R² is hydrogen or hydroxy;
R³ is hydroxy;
R⁴ and R⁵ are independently hydrogen or methyl, and R⁶ is 1-phenylethyl, or R⁵ and R⁶ taken together with the nitrogen atom to which they are connected are N,N-diethyl, N,N-dipropyl or N,N-dibutyl group, or R⁵ and R⁶ taken together with the nitrogen atom to which they are connected form N,N-1″,4″-tetramethylene, N,N-3″-oxa-1″,5″-pentamethylene, N,N-1″,5″-pentamethylene, N,N-1″,6″-hexamethylene, N,N-1″,7″-heptamethylene, N,N-3″-methylaza-1″,5″-pentamethylene, or 1-indolinyl; and
X is HCl, or is absent, wherein when X is HCl the compound is a hydrochloride salt and when X is absent the compound is a free base.

2. The method of claim 1, wherein R³ is axial or equatorial.

3. The method of claim 1, wherein the compound of Formula 1 or Formula 2 is:
3′-deamino-3′-(N,N-1″,4″-tetramethyleneformamidino)-daunorubicin hydrochloride;
3′-deamino-3′-(N,N-1″,4″-tetramethyleneformamidino)-daunorubicin;
3′-deamino-3′-(N,N-3″-oxa-1″,5″-pentamethyleneformamidino)-daunorubicin hydrochloride;
3′-deamino-3′-(N,N-3″-oxa-1″,5″-pentamethyleneformamidino)-daunorubicin;
3′-deamino-3′-(N,N-1″,5″-pentamethyleneformamidino)-daunorubicin hydrochloride;
3′-deamino-3′-(N,N-1″,5″-pentamethyleneformamidino)-daunorubicin;
3′-deamino-3′-(N,N-1″,6″-hexamethyleneformamidino)-daunorubicin hydrochloride;
3′-deamino-3′-(N,N-3″-methylaza-1″,5″-pentamethyleneformamidino)-daunorubicin hydrochloride;
3′-deamino-3′-(N,N-3″-methylaza-1″,5″-pentamethyleneformamidino)-daunorubicin;
3′-deamino-3′-(N,N-o-ethylenephenyleneformamidino)-daunorubicin hydrochloride;
3′-deamino-3′-(N,N-o-ethylenephenyleneformamidino)-daunorubicin;
3′-deamino-3′-(N,N-diethylformamidino)-daunorubicin hydrochloride;
3′-deamino-3′-(N,N-diethylformamidino)-daunorubicin;
3′-deamino-3′-(N,N-dipropylformamidino)-daunorubicin hydrochloride;
3′-deamino-3′-(N,N-dipropylformamidino)-daunorubicin;
3′-deamino-3′-(N,N-dibutylformamidino)-daunorubicin hydrochloride;
3′-deamino-3′-(N,N-dibutylformamidino)-daunorubicin;
3′-deamino-3′-(N,N-1″,4″-tetramethyleneacetamidino)-daunorubicin hydrochloride;
3′-deamino-3′-(N,N-1″,4″-tetramethyleneacetamidino)-daunorubicin;
3′-deamino-3′-(N,N-3″-oxa-1″,5″-pentamethyleneacetamidino)-daunorubicin hydrochloride;
3′-deamino-3′-(N,N-3″-oxa-1″,5″-pentamethyleneacetamidino)-daunorubicin;
3′-deamino-3′-(N,N-1″,5″-pentamethyleneacetamidino)-daunorubicin hydrochloride;
3′-deamino-3′-(N,N-1″,5″-pentamethyleneacetamidino)-daunorubicin;
3′-deamino-3′-(N,N-1″,6″-hexamethyleneacetamidino)-daunorubicin hydrochloride;
3′-deamino-3′-(N,N-3″-methylaza-1″,5″-pentamethyleneacetamidino)-daunorubicin hydrochloride;
3′-deamino-3′-(N,N-3″-methylaza-1″,5″-pentamethyleneacetamidino)-daunorubicin;
3′-deamino-3′-(N,N-o-ethylenephenyleneacetamidino)-daunorubicin hydrochloride;
3′-deamino-3′-(N,N-o-ethylenephenylenacetamidino)-daunorubicin;
3′-deamino-3′-(N,N-diethylacetamidino)-daunorubicin hydrochloride;
3′-deamino-3′-(N,N-diethylacetamidino)-daunorubicin;
3′-deamino-3′-(N,N-dipropylacetamidino)-daunorubicin hydrochloride;
3′-deamino-3′-(N,N-dipropylacetamidino)-daunorubicin;
3′-deamino-3′-(N,N-dibutylacetamidino)-daunorubicin hydrochloride;
3′-deamino-3′-(N,N-dibutylacetamidino)-daunorubicin;
3′-deamino-3′-(N,N-1″,4″-tetramethyleneformamidino)-doxorubicin hydrochloride;
3′-deamino-3′-(N,N-1″,4″-tetramethyleneformamidino)-doxorubicin;
3′-deamino-3′-(N,N-3″-oxa-1″,5″-pentamethyleneformamidino)-doxorubicin hydrochloride;
3′-deamino-3′-(N,N-3″-oxa-1″,5″-pentamethyleneformamidino)-daunorubicin;
3′-deamino-3′-(N,N-1″,5″-pentamethyleneformamidino)-doxorubicin hydrochloride;
3′-deamino-3′-(N,N-1″,5″-pentamethyleneformamidino)-doxorubicin;
3′-deamino-3′-(N,N-1″,6″-hexamethyleneformamidino)-doxorubicin hydrochloride;
3′-deamino-3′-(N,N-3″-methylaza-1″,5″-pentamethyleneformamidino)-doxorubicin hydrochloride;
3′-deamino-3′-(N,N-3″-methylaza-1″,5″-pentamethyleneformamidino)-doxorubicin;
3′-deamino-3′-(N,N-o-ethylenephenyleneformamidino)-doxorubicin hydrochloride;
3′-deamino-3′-(N,N-o-ethylenephenyleneformamidino)-doxorubicin;
3′-deamino-3′-(N,N-diethylformamidino)-doxorubicin hydrochloride;
3′-deamino-3′-(N,N-diethylformamidino)-doxorubicin;
3′-deamino-3′-(N,N-dipropylformamidino)-doxorubicin hydrochloride;
3′-deamino-3′-(N,N-dipropylformamidino)-doxorubicin;
3′-deamino-3′-(N,N-dibutylformamidino)-doxorubicin hydrochloride;
3′-deamino-3′-(N,N-dibutylformamidino)-doxorubicin;
3′-deamino-3′-(N,N-1″,4″-tetramethyleneacetamidino)-doxorubicin hydrochloride;
3′-deamino-3′-(N,N-1″,4″-tetramethyleneacetamidino)-doxorubicin;
3′-deamino-3′-(N,N-3″-oxa-1″,5″-pentamethyleneacetamidino)-doxorubicin hydrochloride;
3′-deamino-3′-(N,N-3″-oxa-1″,5″-pentamethyleneacetamidino)-doxorubicin;
3′-deamino-3′-(N,N-1″,5″-pentamethyleneacetamidino)-doxorubicin hydrochloride;
3′-deamino-3′-(N,N-1″,5″-pentamethyleneacetamidino)-doxorubicin;
3′-deamino-3′-(N,N-1″,6″-hexamethyleneacetamidino)-doxorubicin hydrochloride;
3′-deamino-3′-(N,N-3″-methylaza-1″,5″-pentamethyleneacetamidino)-doxorubicin hydrochloride;
3′-deamino-3′-(N,N-3″-methylaza-1″,5″-pentamethyleneacetamidino)-doxorubicin;
3′-deamino-3′-(N,N-o-ethylenephenyleneacetamidino)-doxorubicin hydrochloride;
3′-deamino-3′-(N,N-o-ethylenephenyleneacetamidino)-doxorubicin;
3′-deamino-3′-(N,N-diethylacetamidino)-doxorubicin hydrochloride;
3′-deamino-3′-(N,N-diethylacetamidino)-doxorubicin;
3′-deamino-3′-(N,N-dipropylacetamidino)-doxorubicin hydrochloride;
3′-deamino-3′-(N,N-dipropylformamidino)-doxorubicin;
3′-deamino-3′-(N,N-dibutylacetamidino)-doxorubicin hydrochloride;
3′-deamino-3′-(N,N-dibutylacetamidino)-doxorubicin;
3′-deamino-3′-(N,N-1″,4″-tetramethyleneformamidino)-epidaunorubicin hydrochloride;
3′-deamino-3′-(N,N-1″,4″-tetramethyleneformamidino)-epidaunorubicin;
3′-deamino-3′-(N,N-3″-oxa-1″,5″-pentamethyleneformamidino)-epidaunorubicin hydrochloride;
3′-deamino-3′-(N,N-3″-oxa-1″,5″-pentamethyleneformamidino)-epidaunorubicin;
3′-deamino-3′-(N,N-1″,5″-pentamethyleneformamidino)-epidaunorubicin hydrochloride;
3′-deamino-3′-(N,N-1″,5″-pentamethyleneformamidino)-epidaunorubicin;
3′-deamino-3′-(N,N-1″,6″-hexamethyleneformamidino)-epidaunorubicin hydrochloride;
3′-deamino-3′-(N,N-3″-methylaza-1″,5″-pentamethyleneformamidino)-epidaunorubicin hydrochloride;
3′-deamino-3′-(N,N-3″-methylaza-1″,5″-pentamethyleneformamidino)-epidaunorubicin;
3′-deamino-3′-(N,N-o-ethylenephenyleneformamidino)-epidaunorubicin hydrochloride;
3′-deamino-3′-(N,N-o-ethylenephenyleneformamidino)-epidaunorubicin;
3′-deamino-3′-(N,N-diethylformamidino)-epidaunorubicin hydrochloride;
3′-deamino-3′-(N,N-diethylformamidino)-epidaunorubicin;
3′-deamino-3′-(N,N-dipropylformamidino)-epidaunorubicin hydrochloride;
3′-deamino-3′-(N,N-dipropylformamidino)-epidaunorubicin;
3′-deamino-3′-(N,N-dibutylformamidino)-epidaunorubicin hydrochloride;
3′-deamino-3′-(N,N-dibutylformamidino)-epidaunorubicin;
3′-deamino-3′-[N-methyl-N-(1″-phenylethyl)-formamidino]-epidaunorubicin hydrochloride;
3′-deamino-3′-(N,N-1″,4″-tetramethyleneacetamidino)-epidaunorubicin hydrochloride,
3′-deamino-3′-(N,N-1″,4″-tetramethyleneacetamidino)-epidaunorubicin;
3′-deamino-3′-(N,N-3″-oxa-1″,5″-pentamethyleneacetamidino)-epidaunorubicin hydrochloride;
3′-deamino-3′-(N,N-3″-oxa-1″,5″-pentamethyleneacetamidino)-epidaunorubicin;
3′-deamino-3′-(N,N-1″,5″-pentamethyleneacetamidino)-epidaunorubicin hydrochloride;
3′-deamino-3′-(N,N-1″,5″-pentamethyleneacetamidino)-epidaunorubicin;
3′-deamino-3′-(N,N-1″,6″-hexamethyleneacetamidino)-epidaunorubicin hydrochloride;
3′-deamino-3′-(N,N-3″-methylaza-1″,5″-pentamethyleneacetamidino)-epidaunorubicin hydrochloride;
3′-deamino-3′-(N,N-3″-methylaza-1″,5″-pentamethyleneacetamidino)-epidaunorubicin;
3′-deamino-3′-(N,N-o-ethylenephenyleneacetamidino)-epidaunorubicin hydrochloride;
3′-deamino-3′-(N,N-o-ethylenephenyleneacetamidino)-epidaunorubicin;
3′-deamino-3′-(N,N-diethylacetamidino)-epidaunorubicin hydrochloride;
3′-deamino-3′-(N,N-diethylacetamidino)-epidaunorubicin;
3′-deamino-3′-(N,N-dipropylacetamidino)-epidaunorubicin hydrochloride;
3′-deamino-3′-(N,N-dipropylacetamidino)-epidaunorubicine;
3′-deamino-3′-(N,N-dibutylacetamidino)-epidaunorubicin hydrochloride;
3′-deamino-3′-(N,N-dibutylacetamidino)-epidaunorubicin;
3′-deamino-3′-[N-methyl-N-(1″-phenylethyl)-acetamidino]-epidaunorubicin hydrochloride;
3′-deamino-3′-(N,N-1″,4″-tetramethyleneformamidino)-epidoxorubicin hydrochloride;
3′-deamino-3′-(N,N-1″,4″-tetramethyleneformamidino)-epidoxorubicin;
3′-deamino-3′-(N,N-3″-oxa-1″,5″-pentamethyleneformamidino)-epidoxorubicin hydrochloride;
3′-deamino-3′-(N,N-3″-oxa-1″,5″-pentamethyleneformamidino)-epidoxorubicin;
3′-deamino-3′-(N,N-1″,5″-pentamethyleneformamidino)-epidoxorubicin hydrochloride;
3′-deamino-3′-(N,N-1″,5″-pentamethyleneformamidino)-epidoxorubicin;
3′-deamino-3′-(N,N-1″,6″-hexamethyleneformamidino)-epidoxorubicin hydrochloride;
3′-deamino-3′-(N,N-3″-methylaza-1″,5″-pentamethyleneformamidino)-epidoxorubicin hydrochloride;
3′-deamino-3′-(N,N-3″-methylaza-1″,5″-pentamethyleneformamidino)-epidoxorubicin;
3′-deamino-3′-(N,N-o-ethylenephenyleneformamidino)-epidoxorubicin hydrochloride;
3′-deamino-3′-(N,N-o-ethylenephenyleneformamidino)-epidoxorubicin;
3′-deamino-3′-(N,N-diethylformamidino)-epidoxorubicin hydrochloride;
3′-deamino-3′-(N,N-diethylformamidino)-epidoxorubicin;
3′-deamino-3′-(N,N-dipropylacetamidino)-epidoxorubicin hydrochloride;
3′-deamino-3′-(N,N-dipropylacetamidino)-epidoxorubicin;
3′-deamino-3′-(N,N-dibutylacetamidino)-epidoxorubicin hydrochloride;
3′-deamino-3′-(N,N-dibutylacetamidino)-epidoxorubicin;
3′-deamino-3′-[N-methyl-N-(1″-phenylethyl)-formamidino]-epidoxorubicin hydrochloride;
3′-deamino-3′-(N,N-1″,4″-tetramethyleneacetamidino)-epidoxorubicin hydrochloride;
3′-deamino-3′-(N,N-1″,4″-tetramethyleneacetamidino)-epidoxorubicin;
3′-deamino-3′-(N,N-3″-oxa-1″,5″-pentamethyleneacetamidino)-epidoxorubicin hydrochloride;
3′-deamino-3′-(N,N-3″-oxa-1″,5″-pentamethyleneacetamidino)-epidoxorubicin;
3′-deamino-3′-(N,N-1″,5″-pentamethyleneacetamidino)-epidoxorubicin hydrochloride;
3′-deamino-3′-(N,N-1″,5″-pentamethyleneacetamidino)-epidoxorubicin;
3′-deamino-3′-(N,N-1″,6″-hexamethyleneacetamidino)-epidoxorubicin hydrochloride;
3′-deamino-3′-(N,N-1″,7″-heptamethyleneacetamidino)-epidoxorubicin hydrochloride;
3′-deamino-3′-(N,N-3″-methylaza-1″,5″-pentamethyleneacetamidino)-epidoxorubicin hydrochloride;
3′-deamino-3′-(N,N-3″-methylaza-1″,5″-pentamethyleneacetamidino)-epidoxorubicin;
3′-deamino-3′-(N,N-o-ethylenephenyleneacetamidino)-epidoxorubicin hydrochloride;
3′-deamino-3′-(N,N-o-ethylenephenyleneacetamidino)-epidoxorubicin;
3′-deamino-3′-(N,N-diethylacetamidino)-epidoxorubicin hydrochloride;
3′-deamino-3′-(N,N-diethylacetamidino)-epidoxorubicin;
3′-deamino-3′-(N,N-dipropylacetamidino)-epidoxorubicin hydrochloride;
3′-deamino-3′-(N,N-dipropylacetamidino)-epidoxorubicin;
3′-deamino-3′-(N,N-dibutylacetamidino)-epidoxorubicin hydrochloride;
3′-deamino-3′-(N,N-dibutylacetamidino)-epidoxorubicin;
3′-deamino-3′-[N-methyl-N-(1″-phenylethyl)-acetamidino]-epidoxorubicin hydrochloride;
3′-deamino-3′-N,4′-O-methylidenedaunorubicin;
3′-deamino-3′-N,4′-O-methylidenedoxorubicin;
3′-deamino-3′-N,4′-O-ethylidenedaunorubicin; or 3′-deamino-3′-N,4′-O-ethylidenedoxorubicin.

4. The method of claim 1, wherein the pharmaceutical composition further comprises epidoxorubicin, doxorubicin, epidaunorubicin, or daunorubicin, or another antiviral drug.

5. The method of claim 1, wherein the pharmaceutical composition further comprises a second compound of Formula 1 or Formula 2, wherein R¹, R², R³, R⁴, R⁵, R⁶ and X are as defined above.

6. A method for inhibiting the replication of hepatitis C virus comprising exposing the virus to a hepatitis C viral NS3 protease inhibiting amount of:
(a) a compound of Formula 1 or Formula 2, or
(b) a pharmaceutical composition comprising a pharmaceutically-acceptable carrier, diluent or excipient and a compound of Formula 1 or Formula 2, 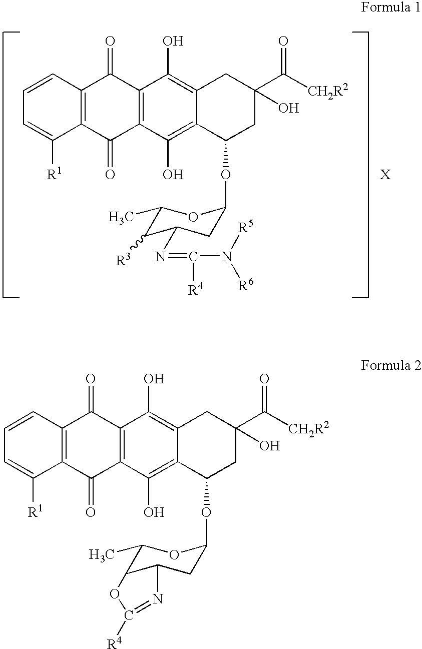
wherein
R¹ is hydrogen or methoxy;
R² is hydrogen or hydroxy;
R³ is hydroxy;
R⁴ and R⁵ are independently hydrogen or methyl, and R⁶ is 1-phenylethyl, or R⁵ and R⁶ taken together with the nitrogen atom to which they are connected are N,N-diethyl, N,N-dipropyl or N,N-dibutyl group, or R⁵ and R⁶ taken together with the nitrogen atom to which they are connected form N,N-1″,4″-tetramethylene, N,N-3″-oxa-1″,5″-pentamethylene, N,N-1″,5″-pentamethylene, N,N-1″,6″-hexamethylene, N,N-1″,7″-heptamethylene, N,N-3″-methylaza-1″,5″-pentamethylene, or 1-indolinyl; and
X is HCl, or is absent, wherein when X is HCl the compound is a hydrochloride salt and when X is absent the compound is a free base.

7. The method of claim 6, wherein the pharmaceutical composition further comprises epidoxorubicin, doxorubicin, epidaunorubicin, or daunorubicin, or another antiviral drug.

8. The method of claim 6, wherein the pharmaceutical composition further comprises a second compound of Formula 1 or Formula 2, wherein R¹, R², R³, R⁴, R⁵, R⁶ and X are as defined above.

9. A pharmaceutical composition exhibiting an antihelicase activity comprising a pharmaceutically-acceptable carrier, diluent or excipient, and a compound of Formula 1 or Formula 2, 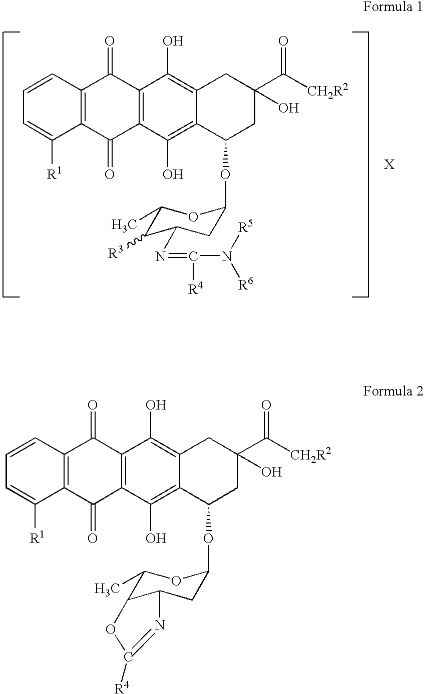
wherein
R¹ is hydrogen or methoxy;
R² is hydrogen or hydroxy;
R³ is hydroxy;
R⁴ and R⁵ are independently hydrogen or methyl, and R⁶ is 1-phenylethyl, or R⁵ and R⁶ taken together with the nitrogen atom to which they are connected are N,N-diethyl, N,N-dipropyl or N,N-dibutyl group, or R⁵ and R⁶ taken together with the nitrogen atom to which they are connected form N,N-1″,4″-tetramethylene, N,N-3″-oxa-1″,5″-pentamethylene, N,N-1″,5″-pentamethylene, N,N-1″,6″-hexamethylene, N,N-1″,7″-heptamethylene, N,N-3″-methylaza-1″,5″-pentamethylene, or 1-indolinyl; and
X is HCl, or is absent, wherein when X is HCl the compound is a hydrochloride salt and when X is absent the compound is a free base.

10. The pharmaceutical composition of claim 9, comprising further a second compound of Formula 1 or Formula 2, 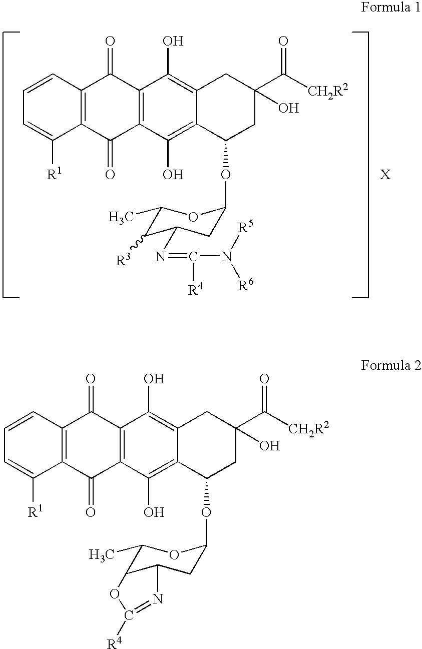
wherein
R¹ is hydrogen or methoxy;
R² is hydrogen or hydroxy;
R³ is hydroxy;
R⁴ and R⁵ are independently hydrogen or methyl, and R⁶ is 1-phenylethyl, or R⁵ and R⁶ taken together with the nitrogen atom to which they are connected are N,N-diethyl, N,N-dipropyl or N,N-dibutyl group, or R⁵ and R⁶ taken together with the nitrogen atom to which they are connected form N,N-1″,4″-tetramethylene, N,N-3″-oxa-1″,5″-pentamethylene, N,N-1″,5″-pentamethylene, N,N-1″,6″-hexamethylene, N,N-1″,7″-heptamethylene, N,N-3″-methylaza-1″,5″-pentamethylene, or 1-indolinyl; and
X is HCl, or is absent, wherein when X is HCl the compound is a hydrochloride salt and when X is absent the compound is a free base.

11. The pharmaceutical composition of claim 9, comprising further epidoxorubicin, doxorubicin, epidaunorubicin, or daunorubicin.

12. The pharmaceutical composition of claim 10, comprising further another antiviral drug.

13. The pharmaceutical composition of claim 11, comprising further another antiviral drug.
